(19) United States
(12) Patent Application Publication
(10) Pub. No.: US 2013/0197077 A1
(43) Pub. Date: Aug. 1, 2013

(54) Bicyclic Furanones with Low Toxicity for Microbial Control

(71) Applicant: Syracuse University, Syracuse, NY (US)

(72) Inventors: Yan-Yeung Luk, Manlius, NY (US); Sijie Yang, Syracuse, NY (US)

(73) Assignee: SYRACUSE UNIVERSITY, Syracuse, NY (US)

(21) Appl. No.: 13/750,106

(22) Filed: Jan. 25, 2013

Related U.S. Application Data
(60) Provisional application No. 61/591,080, filed on Jan. 26, 2012.

(51) Int. Cl. A01N 43/12; C07D 307/83; C07D 307/93; C07D 307/935

(52) U.S. Cl. CPC A01N 43/12; C07D 307/935; C07D 307/83; C07D 307/93
USPC 514/470; 549/311; 549/302

(57) ABSTRACT

A class of bicyclic brominated furanone structures that have reduced toxicity and high activity for inhibiting biofilm formation and quorum sensing by microbes. The molecules have two fused cyclic alkyl groups that provide a structural framework that retain one or more bromine groups on the structure. The bicyclic furanones have reduced toxicity to mammalian cells as compared to other brominated furanones but retain the ability to inhibit biofilm formation in bacterial populations.

### CROSS-REFERENCE TO RELATED APPLICATIONS

The present application claims priority to U.S. Provisional Application No. 61/591,080, filed on Jan. 26, 2012, hereby incorporated by reference.

### STATEMENT REGARDING FEDERALLY SPONSORED RESEARCH OR DEVELOPMENT

This invention was made with government support under CAREER contract no. 0845686 awarded by the National Science Foundation (NSF). The government has certain rights in the invention.

### BACKGROUND OF THE INVENTION

1. Field of the Invention

The present invention relates to antimicrobial compounds and, more specifically, to a new class of furanones having reduced toxicity while inhibiting biofilm formation.

2. Description of the Related Art

Many diseases are related to the persistent presence of films hosting bacteria. Brominated furanones are a class of molecules that are effective for controlling bacteria behavior and, in particular, inhibiting the formation of biofilms, and thus could be used to control infectious diseases and other biofilm related problems. One major setback with known brominated furanones, however, is their toxicity. Accordingly, there is a need for compounds that inhibit biofilm formation but are less toxic than known brominated furanones.

### BRIEF SUMMARY OF THE INVENTION

The present invention involves a new class of furanone structures that has reduced toxicity and high activity for inhibiting biofilm formation by microbials. This technology describes the structure and synthesis of a new class of bicyclic structures that reduce the bromine content, thus reducing the toxicity of the molecules, while retaining the activity for inhibiting biofilm formation by bacteria. This class of molecules is built using two fused cyclic alkyl groups in the molecules that provide a structural framework that can optionally retain one or more bromine groups on the structure. Experimental testing indicates that these new structures are less toxic than known brominated furanones. However, the new class of bicyclic compounds continues to inhibit biofilm formation.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a series of chemical diagrams of the bicyclic brominated furanones according to the present invention;

FIG. 2A is a chemical diagram for an exemplary bicyclic brominated furanone according to the present invention that was tested and compared against two exemplary conventional brominated furanones as seen in FIG. 2B;

FIG. 3A-E are representative confocal laser scanning microscopy (CLSM) images of biofilm formed by E. coli RP437 (pRSH103) in the absence and presence of the exemplary bicyclic brominated furanones or an exemplary conventional brominated furanone at 200 μM;

FIG. 4 is a graph of the quantification of biofilm formation by E. coli RP437 (pRSH103) in the absence and presence of 200 μM brominated furanones;

FIG. 5 is a graph comparing the biofilm inhibiting and dispersing activity of the exemplary bicyclic brominated furanones according to the present invention verses an exemplary conventional brominated furanone using a colorimetric assay employing crystal violet;

FIG. 6A-D are representative confocal laser scan microscopy (CLSM) images of biofilm formed by PA01-GFP (expresses green fluorescence on plasmid pSMC2) in the absence and presence of the exemplary bicyclic brominated furanones or an exemplary conventional brominated furanone at 400 μM;

FIG. 7 is a graph of the quantification of biofilm formation by PA01-GFP (pSMC2) in the absence and presence of 400 μM brominated furanones;

FIG. 8 is a graph of the effect of brominated furanones on las quorum sensing in P. aeruginosa PAO-JP2 (plasI-LVAgfp) measured by GFP expression in the presence of 1 μM natural autoinducer PAH alone (control) or 1 μM PAI1 plus various concentration of bicyclic-BFs or BF8, where fluorescence signals were corrected for cell density by dividing by OD₆₀₀ of cell culture and the results were normalized to the control;

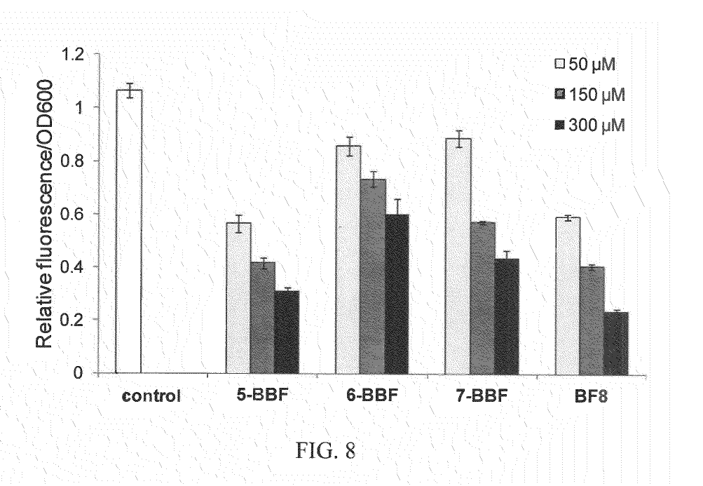

FIG. 9 is a graph of the effect of brominated furanones on rhl quorum sensing in P. aeruginosa PAO-JP2 (prhlI-LVAgfp) measured by GFP expression in the presence of 1 μM natural autoinducer PAI1 and 10 μM PAI2 alone (control) or 1 μM PAI1 and 10 μM PAI2 plus various concentration of bicyclic-BFs or BF8, where fluorescence signals were corrected for cell density by dividing by OD₆₀₀ of cell culture and the results were normalized to the control;

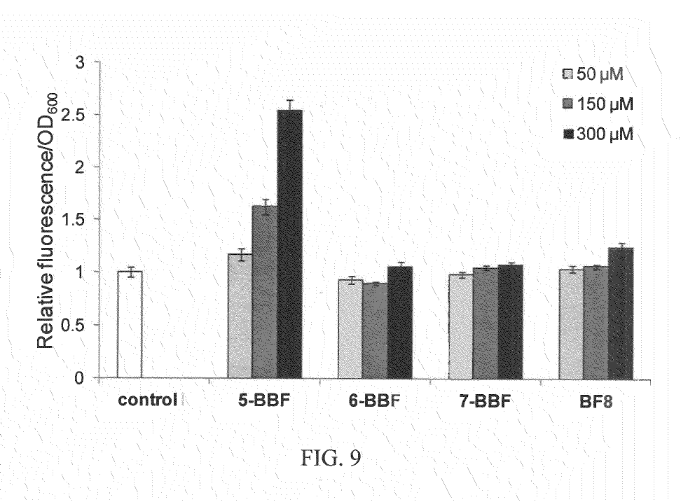

FIG. 10 is a graph comparing the cytotoxicity of the exemplary bicyclic brominated furanones according to the present invention verses an exemplary conventional brominated furanone relative to E. Coli RP437;

FIG. 11 is a graph comparing the cytotoxicity of the exemplary bicyclic brominated furanones according to the present invention verses an exemplary conventional brominated furanone relative to P. aeruginosa PAO1;

FIG. 12 is a graph comparing the cytotoxicity of the exemplary bicyclic brominated furanones according to the present invention verses an exemplary conventional brominated furanone relative to mammalian cell line 3T3 mouse fibroblasts;

FIG. 13 is a graph comparing the cytotoxicity of the exemplary bicyclic brominated furanones according to the present invention verses an exemplary conventional brominated furanone relative to mammalian cell line SK-N-SH human neuroblastoma;

FIG. 14 is a chemical diagram of the general process for synthesizing bicyclic brominated furanones according to the present invention.

### DETAILED DESCRIPTION OF THE INVENTION

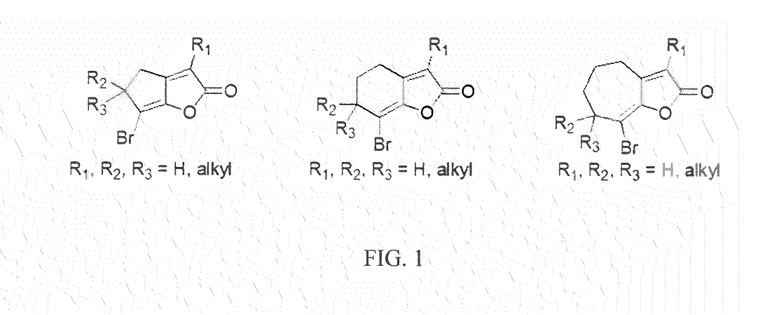

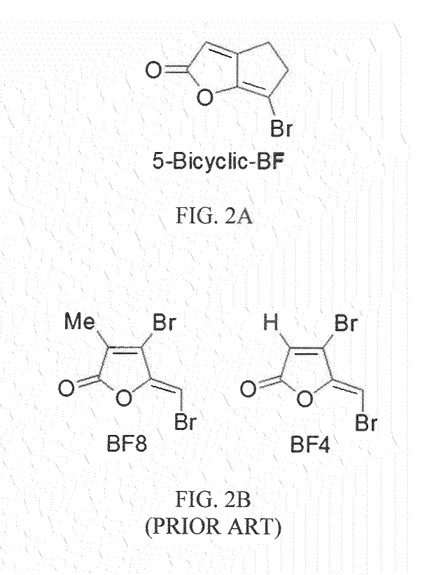

Referring now to the drawings, wherein like reference numerals refer to like parts throughout, there is seen in FIG. 1 the chemical structures for a new class of bicyclic brominated furanones according to the present invention. As seen in FIG. 2A, exemplary bicyclic brominated furanones (referred to as Bicyclic-BFs) were tested and compared against two conventional brominated furanones (referred to as BF8 and BF4) to evaluate the toxicity and effectiveness of the new class of compounds, as seen in FIG. 2B.

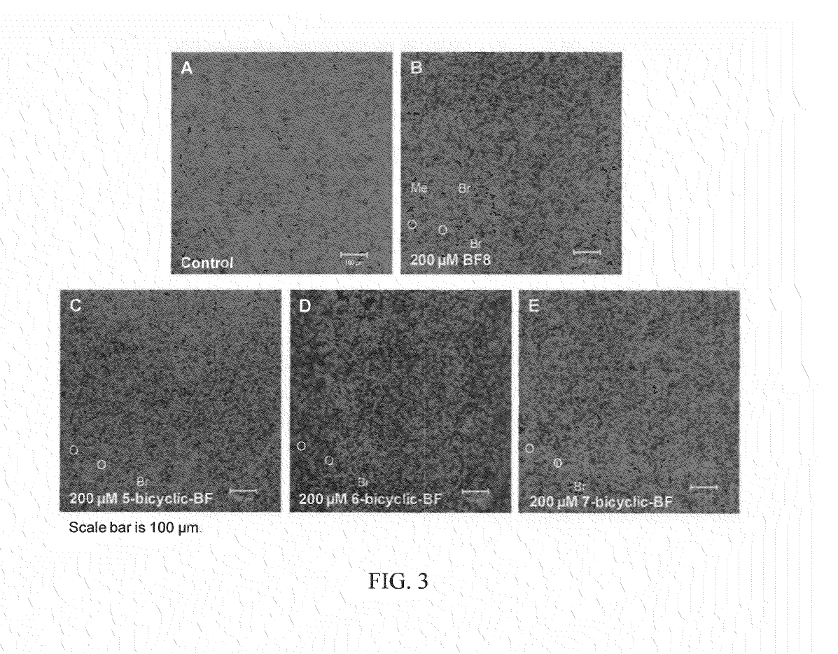

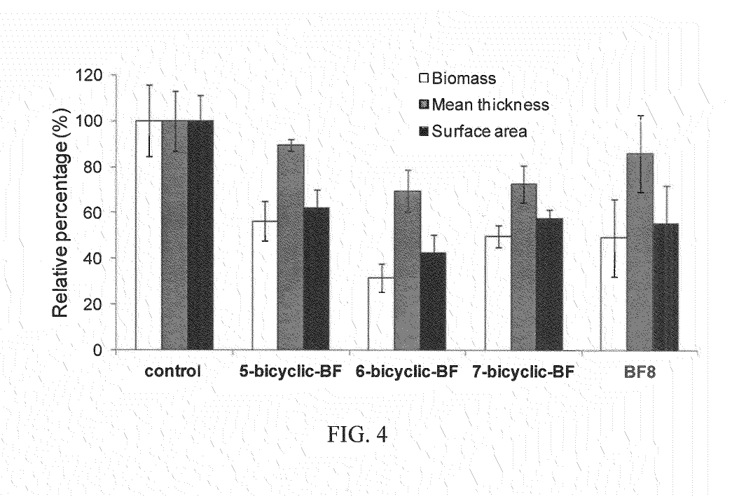

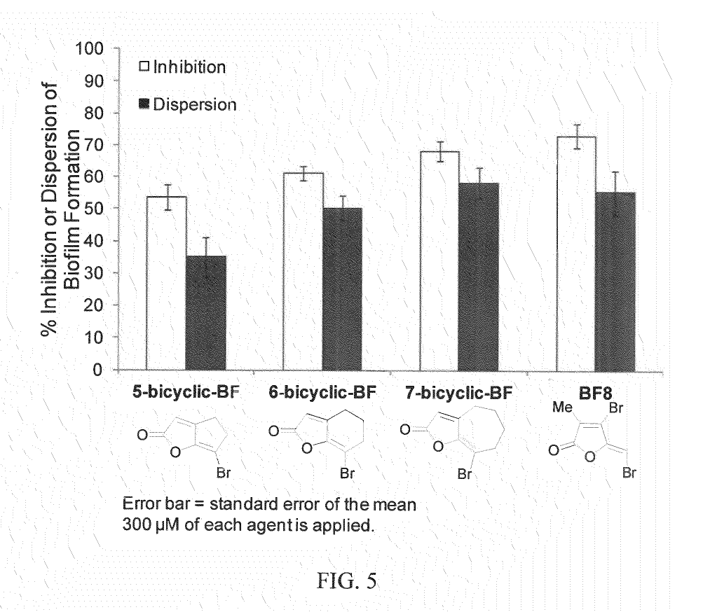

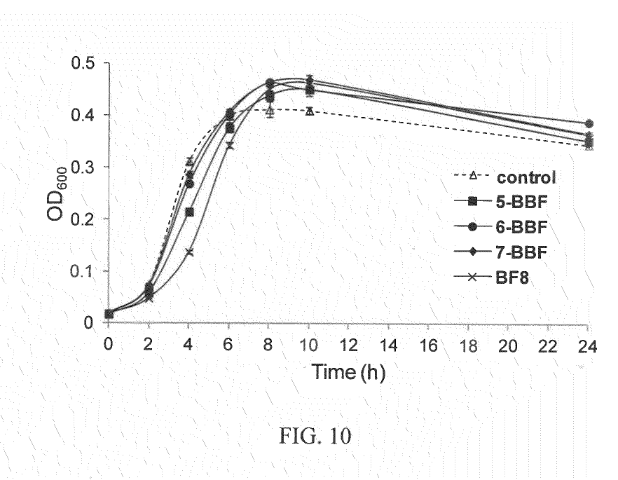

Referring to FIG. 3-5, Bicyclic-BFs inhibit biofilm formation of E. coli RP437 as effectively as the more toxic BF8. At the same time, as seen in FIG. 10, Bicyclic-BF is not as toxic to the growth of E. coli RP437.

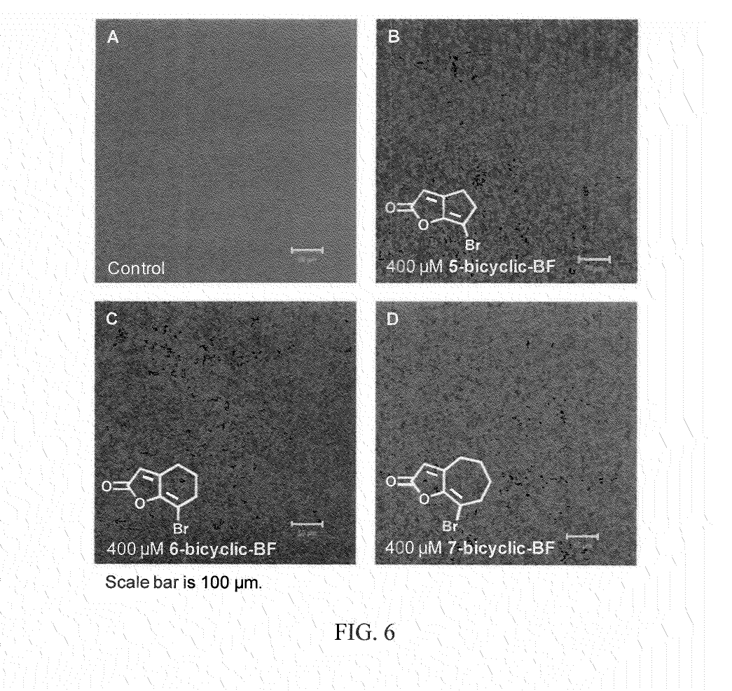

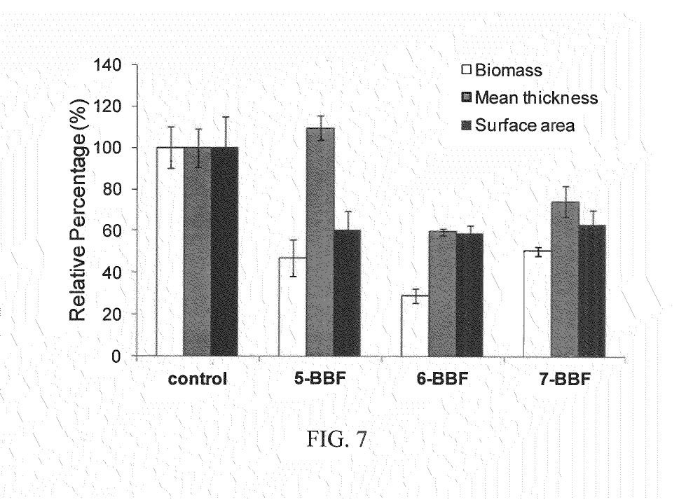

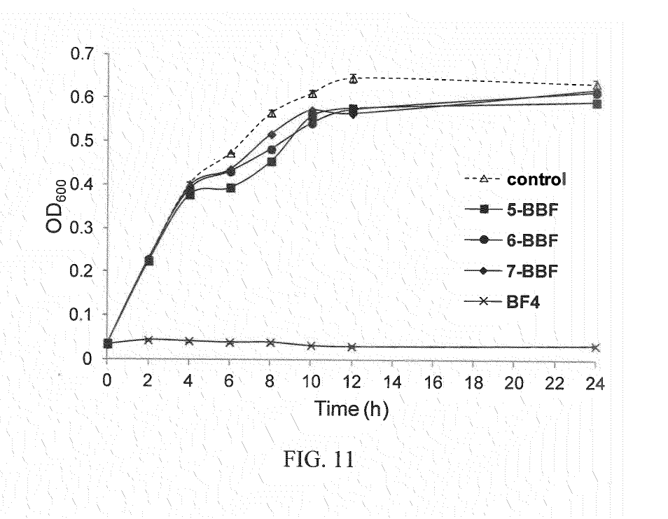

Referring to FIG. 6-7, Bicyclic-BFs inhibit biofilm formation of P. aeruginosa PAO1 slightly less effectively than the more toxic BF4. At the same time, as seen in FIG. 11, Bicyclic-BF is not as toxic to the growth of P. aeruginosa PAO1.

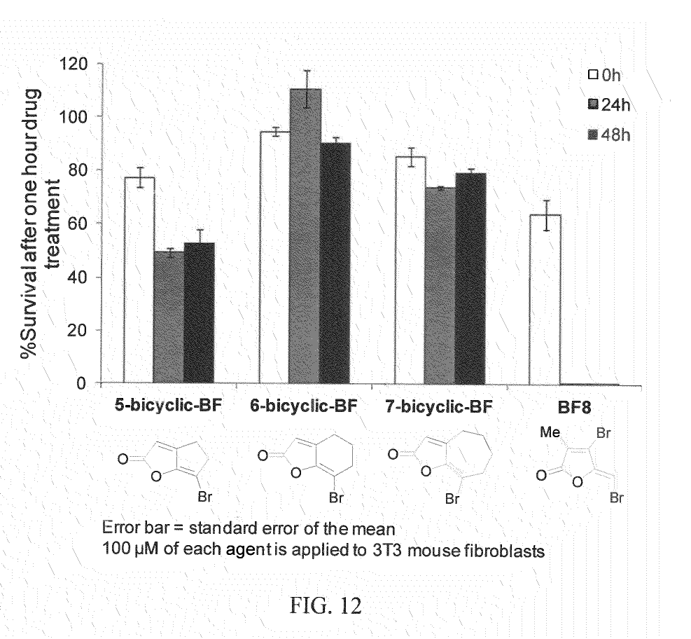

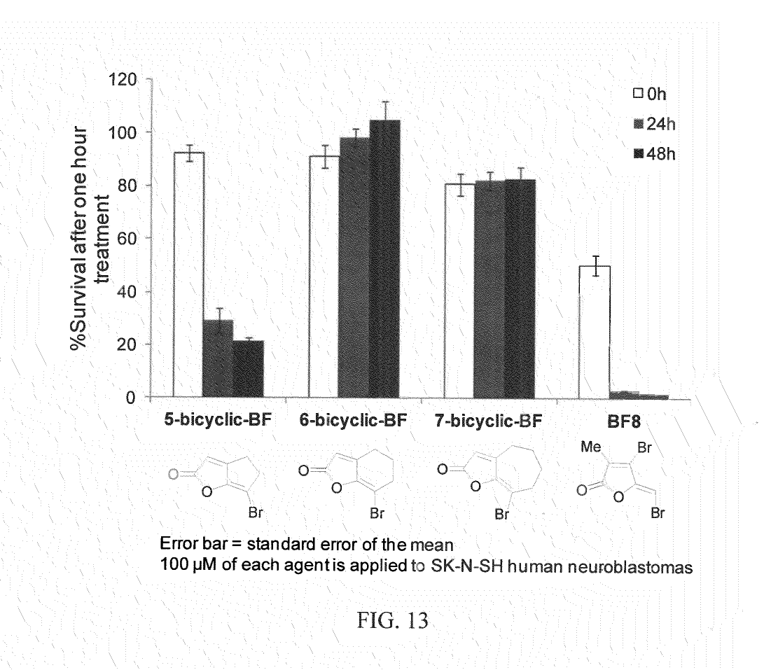

As further seen in FIGS. 12 and 13, Bicyclic-BFs also exhibit less toxicity to mammalian cells. In this test, the mammalian cells used were 3T3 mouse fibroblasts and SK-N-SH human neuroblastomas, and 0 h, 24 h, and 48 h, refer to recovery time of cells after removal of drugs. At the beginning of the experiment, the survival of the cells is assumed to be 100 percent. The survival percentage is calculated using the following formula:

Survival (%)=(OD₄₅₀ sample−OD₄₅₀ medium)/(OD₄₅₀ control−OD₄₅₀ medium)×100.

The sample OD was obtained from the wells containing cells and drugs, and the control OD was obtained from wells containing cells+1% DMSO. BF8, 5-, and 7-bicylic-BFs are cytotoxic to mammalian cell 3T3 mouse fibroblasts and SK-N-SH human neuroblastomas at 100 μM after 0 h, 24 h, and 48 h of incubation, but 5-, and 7-bicyclic-BF are less cytotoxic than BF8 at 100 μM. 6-Bicyclic-BF is almost noncytotoxic to 3T3 mouse fibroblasts and SK-N-SH human neuroblastomas at 100 μM.

With respect to effectiveness in inhibiting biofilm formation, as seen in FIGS. 3-5, Bicyclic-BFs and BF8 are similar in inhibitory activity toward biofilm formation of E. coli RP437.

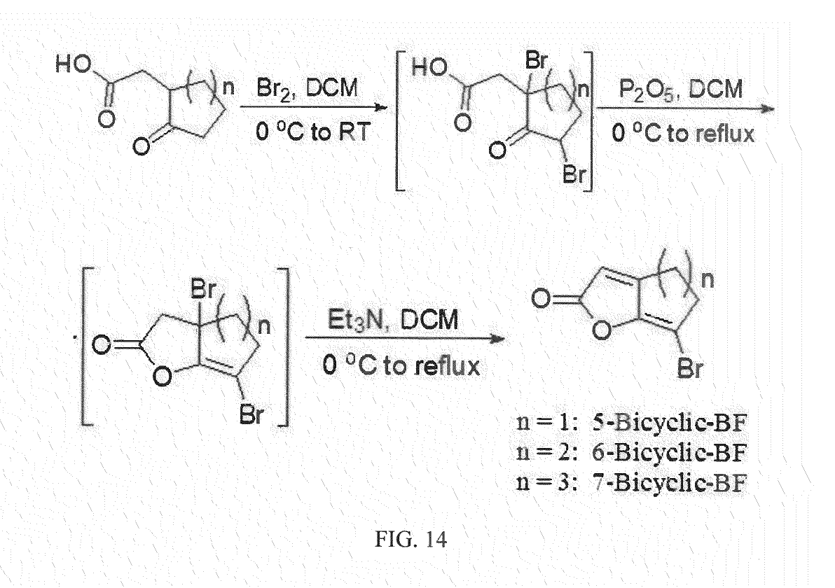

With respect to compound synthesis, the 5-, 6-, and 7-bicyclic-BFs seen in FIG. 1 were prepared using the same synthetic procedure with different starting reactants. Those of skill in the art should appreciate that the other bicyclic brominated furanones can be manufactured from other starting reactants using the same process. Following is an example the general synthesis of this class of molecules using the synthesis of 5-bicyclic-BF, as seen in FIG. 14, as an example. Bromine (0.79 mL, 15 mmol) was added dropwise to a solution of 2-oxocyclopentaneacetic acid (1.1274 g, 7.693 mmol) in anhydrous methylene chloride (7.7 mL). The reaction mixture was stirred for 100 min. The resulting solution was washed with water (10 mL) followed by aqueous 1M Na₂S₂O₃ solution (10 mL) and then extracted with methylene chloride (10 mL×3). The combined organic layer was dried over MgSO₄, filtered and concentrated under reduced pressure. The crude oil was taken up in anhydrous methylene chloride (30 mL) followed by addition of P₂O₄ (2.6455 g, 18.64 mmol). The reaction mixture was stirred under reflux for 2 h. The solid thus formed was filtered off and the filtrate concentrated under reduced pressure. The crude oil was dissolved in methylene chloride (15 mL) and treated with anhydrous Et₃N (1.12 mL, 8.0 mmol) under reflux for 3 h. After cooled to ambient temperature, the reaction mixture was washed with aqueous saturated NH₄Cl and extracted with methylene chloride (10 mL×3). The combined organic layer was dried over MgSO₄, filtered and concentrated under reduced pressure. Flash chromatography (SiO₂, hexane:ethyl acetate, gradient) provided 5-bicyclic-BF as off white solid.

Bicyclic brominated furanones according to the present invention may be used to develop disinfectant sprays or wipes for military use and for domestic use. Bicyclic brominated furanones according to the present invention may also be used as chemical agents for hospital use to reduce infectious diseases, and may be developed into drugs for the treatment of infectious diseases particularly where biofilm formation is problematic.

This class of bicyclic brominated furanones can inhibit biofilm formation by detrimental microbes including, but not limited to, Candida albicans, staphylococcus, E. coli, Pseudomonas aeruginosa, Burkholderia cenocepacia, Mycobacterium avium.

What is claimed is:

1. A bicyclic furanone comprising two fused cyclic alkyl groups and at least one bromine group coupled to the fused cyclic alkyl groups.

2. The bicyclic brominated furanone of claim 1 having the formula 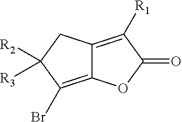
where R₁, R₂, and R₃ comprise a hydrogen atom or an alkyl group.

3. The bicyclic brominated furanone of claim 1 having the formula 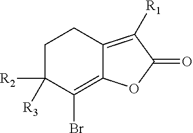
where R₁, R₂, and R₃ comprise a hydrogen atom or an alkyl group.

4. The bicyclic brominated furanone of claim 1 having the formula 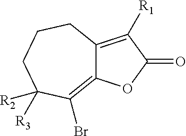
where R₁, R₂, and R₃ comprise a hydrogen atom or an alkyl group.

5. A method of inhibiting the formation of a biofilm in a bacterial population comprising the step of treating the bacterial population with a bicyclic brominated furanone having two fused cyclic alkyl groups and at least one bromine group coupled to the fused cyclic alkyl groups.

6. The method of claim 1, wherein the bicyclic brominated furanone has the formula 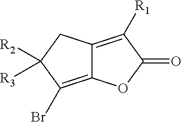
where R₁, R₂, and R₃ comprise a hydrogen atom or an alkyl group.

7. The method of claim 1, wherein the bicyclic brominated furanone has the formula 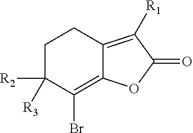
where R₁, R₂, and R₃ comprise a hydrogen atom or an alkyl group.

8. The method of claim 1, wherein the bicyclic brominated furanone has the formula 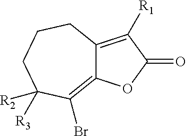
where R₁, R₂, and R₃ comprise a hydrogen atom or an alkyl group.
